(19) United States
(12) Patent Application Publication
(10) Pub. No.: US 2007/0299388 A1
(43) Pub. Date: Dec. 27, 2007

(54) MICROPROJECTION ARRAY APPLICATION WITH MULTILAYERED MICROPROJECTION MEMBER FOR HIGH DRUG LOADING

(75) Inventors: Keith Chan, Brookline, MA (US); Rajan Patel, Menlo Park, CA (US); Peter E. Daddona, Menlo Park, CA (US); Cedric Wright, Mountain View, CA (US); Neha Agarwal, Los Altos Hill, CA (US)

Correspondence Address: WILSON SONSINI GOODRICH & ROSATI & MACROFLUX CORP., 650 PAGE MILL ROAD, PALO ALTO, CA, 94304, US

(73) Assignee: ALZA CORPORATION, Mountain View, CA (US)

(21) Appl. No.: 11/740,258

(22) Filed: Apr. 25, 2007

Related U.S. Application Data
(60) Provisional application No. 60/794,960, filed on Apr. 25, 2006.

(51) Int. Cl. A61M 37/00

(52) U.S. Cl. 604/46

(57) ABSTRACT

A transdermal drug delivery system with microprojections for disrupting a body surface to an individual. At least some of the microprojections arise from a first microprojection layer and at least some of the microprojections arise from a second microprojection layer. The first and second microprojection layers are stacked together.

### CROSS-REFERENCE

This application claims the benefit of U.S. Provisional Application No. 60/794,960, filed Apr. 25, 2006, which application is incorporated herein by reference.

### BACKGROUND OF THE INVENTION

This invention relates to an apparatus and method for applying a microprojection array to the stratum corneum by impact, and more particularly, the invention relates to a microprojection array having high drug loading thereon.

The natural barrier function of the body surface, such as skin, presents a challenge to delivery therapeutics into circulation. Transdermal devices for the delivery of biologically active agents or drugs have been used for maintaining health and therapeutically treating a wide variety of ailments. For example, analgesics, steroids, etc., have been delivered with such devices. Transdermal drug delivery can generally be considered to belong to one of two groups: transport by a “passive” mechanism or by an “active” transport mechanism. In the former, such as drug delivery skin patches, the drug is incorporated in a solid matrix, a reservoir, and/or an adhesive system.

There are various ways to increase transdermal delivery rates. One way to increase the transdermal delivery of agents is to pretreat the skin with, or co-delivering with the beneficial agent, a skin permeation enhancer. A permeation enhancer substance, when applied to a body surface through which the agent is delivered, enhances the transdermal flux of the agent such as by increasing the permselectivity and/or permeability of the body surface, and/or reducing the degradation of the agent.

Another type of transdermal drug delivery is active transport in which the drug flux is driven by various forms of energy. Iontophoresis, for example, is an “active” electrotransport delivery technique that transports solubilized drugs across the skin by an electrical current. The feasibility of this mechanism is constrained by the solubility, diffusion and stability of the drugs, as well as electrochemistry in the device. The transport of the agent is induced or enhanced by the application of an applied electrical potential, which results in the application of electric current, to deliver or enhance delivery of the agent.

However, at the present many drugs and pharmaceutical agents still cannot be efficiently delivered by conventional passive patches or electrotransport systems through intact body surfaces. There is an interest in the percutaneous or transdermal delivery of larger molecules such as peptides and proteins to the human body as increasing number of medically useful peptides and proteins become available in large quantities and pure form. The transdermal delivery of larger molecules such as peptides and proteins still faces significant challenges. In many instances, the rate of delivery or flux of polypeptides through the skin is insufficient to produce a desired therapeutic effect due to their large size and molecular weight. In addition, polypeptides, proteins, and many biologics are easily degraded during and after penetration into the skin, prior to reaching target cells. On the other hand, the passive transdermal flux of many low molecular weight compounds is too limited to be therapeutically effective.

Yet another method to increase transdermal flux (e.g., across skin) is to mechanically penetrate or disrupt the skin. This technique has been mentioned in, for example, U.S. Pat. No. 5,879,326 issued to Godshall, et al., U.S. Pat. No. 3,814,097 issued to Ganderton, et al., U.S. Pat. No. 5,279,544 issued to Gross, et al., U.S. Pat. No. 5,250,023 issued to Lee, et al., U.S. Pat. No. 3,964,482 issued to Gerstel, et al., Reissue 25,637 issued to Kravitz, et al., and PCT Publication Nos, WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365. These devices use piercing elements or microprojections of various shapes and sizes to pierce the outermost layer (i.e., the stratum corneum) of the skin. The microprojections disclosed in these references generally extend perpendicularly from a thin, flat member, such as a pad or sheet. The microprojections in some of these devices are extremely small, some having dimensions (i.e., a microblade length and width) of only about 25-400, and a microblade thickness of only about 5-50μ. Other penetrating elements are hollow needles having diameters of about 10μ or less and lengths of about 50-100μ. These tiny stratum corneum piercing/cutting elements are meant to make correspondingly small microslits/microcuts in the stratum corneum for enhanced transdermal agent delivery or transdermal body analyte sampling therethrough. The perforated skin provides improved flux for sustained agent delivery or sampling through the skin. In many instances, the microslits/microcuts in the stratum corneum have a length of less than 150μ and a width that is substantially smaller than their length.

When microprojection arrays are used to improve delivery or sampling of agents through the skin, consistent, complete, and repeatable microprojection penetration is desired. Microprojection arrays generally have the form of a thin, flat pad or sheet with a plurality of microprojections extending roughly perpendicularly upward and are difficult to handle if they are too big. When an individual manually pushes the microprotrusion array on the skin by hand, the push force may be hard to control and may be uneven across the area of the array. Thus, mechanically actuated devices have been invented to apply a microprojection array to the stratum to effect microprojection skin piercing penetration in a more consistent and repeatable manner. However, even with the help of a mechanical actuator, a large microprojection array is still hard to apply to the body surface since body surfaces are generally not actually flat. Further, large microprojection arrays are inconvenient and uncomfortable for the patient. Because many chemical drugs are not highly potent, to deliver an effective amount of the drug, increasing the drug loading per unit planar area of a microprojection member holding the microprojection array is desirable.

Microprojections for transdermal drug delivery are typically manufactured from a single material layer that has been processed into a plurality of individual microprojections (“array”). Examples of this would include embodiments made from etching a silicon layer, etching/stamping/cutting a metal foil, or molding “sheets” of polymer microprojections. Such microprojection designs and manufacturing methods impose limits in the microprojection design, the total drug loading, and the spatial separation between individual microprojections. Microprojections formed using an etched metal foil cannot be positioned in a way such that the different individual microprojections occupy the same window space. Similarly, microprojections molded within cavities can only be spaced based on the limitations of mold manufacturing.

For example, for microprojection array devices having etched arrays from metallic foil, which microprojections are then formed out-of-plane and surface-coated with drug, the coating is established on each microprojection by wetting the microprojection with a drug formulation as it dips into a drug formulation film. The repeated dipping increases the total drug loading on each tip. However, repeated dipping increases the drug coating profile and the increasing drug coating profile not only hinders skin penetration but also increases the force imparted on the drug coating during skin penetration, thereby increasing the risk of the drug coating sloughing off prior to delivery.

What is needed is a microprojection array that has a higher capacity to hold drug than prior devices. The present invention provides system and methods of making and using such systems in which the microprojection array has a microprojection array that has high drug loading.

### SUMMARY OF THE INVENTION

This invention is related to microprojection systems and methodology that provide a microprojection array for application of the microprojections to the stratum corneum. The microprojection array includes a plurality of microprojections that penetrate the stratum corneum to improve transport of one or more agents across the stratum corneum. The microprojection member has a multilayered base member supporting microprojections. The multilayered base member has at least a first base layer and a second base layer. The first base layer supports a first plurality of microprojections and the second base layer supports a second plurality of microprojections. The first plurality of microprojections and the second plurality of microprojections form a microprojection array for piercing the stratum corneum.

In accordance with an additional aspect of the invention, in a device for drug delivery is a microprojection array with a plurality of stratum corneum piercing microprojections for piercing stratum corneum. The microprojection member has a multilayered base member supporting microprojections. The multilayered base member has at least a first base layer and a second base layer. The first base layer supports a first plurality of microprojections and the second base layer supports a second plurality of microprojections. The first base layer has openings through which microprojections from the second plurality of microprojections can protrude to form the microprojection array with the first plurality of microprojections. The first base layer is integral and continuous with the first plurality of microprojections. Similarly, the second base layer is integral and continuous with the second plurality of microprojections.

In accordance with another aspect of the invention, in a device for drug delivery is a microprojection array with a plurality of stratum corneum piercing microprojections for piercing stratum corneum. The microprojection member has a multilayered base member supporting microprojections. The multilayered base member has at least a first base layer and a second base layer. The first base layer supports a first plurality of microprojections and the second base layer supports a second plurality of microprojections. At least one of the first plurality of microprojections or the second plurality of microprojections extends at a non-perpendicular angle from the first or second base layer, respectively.

In accordance with an additional aspect of the invention, a device for drug delivery includes a microprojection array with a plurality of stratum corneum piercing microprojections for piercing stratum corneum. The multilayered base member has at least a first base layer and a second base layer. The first base layer supports a first plurality of microprojections and the second base layer supports a second plurality of microprojections. The first base layer has openings through which microprojections from the second plurality of microprojections can protrude to form the microprojection array with the first plurality of microprojections. The openings of the first layer and the second layer can be aligned such that the combined first layer and second layer opening is void of base material if viewed from a line normal to a plane of the base layer.

In accordance with an additional aspect of the invention, a device for drug delivery includes a microprojection array with a plurality of stratum corneum piercing microprojections for piercing stratum corneum. The multilayered base member has at least a first base layer and a second base layer. The first base layer supports a first plurality of microprojections and the second base layer supports a second plurality of microprojections. The first base layer has openings through which microprojections from the second plurality of microprojections can protrude to form the microprojection array with the first plurality of microprojections. In this aspect, the base layers are stacked together and rigidly secured together. Additionally, the first base layer and the second base layer can be interference fit together to secure the bases to each other.

In accordance with an additional aspect of the invention, a device for drug delivery includes a microprojection array with a plurality of stratum corneum piercing microprojections for piercing stratum corneum. The multilayered base member has at least a first base layer and a second base layer. The first base layer supports a first plurality of microprojections and the second base layer supports a second plurality of microprojections. At least some of the microprojections of the first plurality of microprojections group with at least some of the microprojections of the second plurality of microprojections. Preferably, a drug coating is coated on at least a portion of the microprojections in the group.

In accordance with additional aspects of the invention, a device for drug delivery includes a microprojection array with a plurality of stratum corneum piercing microprojections for piercing stratum corneum. The microprojection member has a multilayered base member for supporting microprojections and has a first base layer and a second base layer. The first base layer is continuous and integrally supports a first plurality of microprojections. The second base layer is continuous and integrally supports a second plurality of microprojections. The first plurality of microprojections neighbor the second plurality of microprojections and form groups of microprojections in a microprojection array. The microprojection array is coated with a drug coating on at least a portion of the microprojection member. In a further aspect, the microprojection member form a pattern of pairs in the microprojection array.

In further aspects of the present invention, in any of the previously described embodiments, the microprojection member includes at least a first base layer and a second base layer, in which the first base layer has microprojections with shafts in which the shaft length is different from the shaft length of the second plurality of microprojections from the second base layers. In yet another aspect, at least some of the microprojections from a first base layer are positioned in groups with microprojections from a second base layer of the microprojection member and in a group at least one microprojection leans towards another microprojection. In an alternative aspect, the microprojection member consists of a first plurality of microprojections that extend from the first base layer at a first angle and a second plurality of microprojections that extend from the second base layer at a second angle. The microprojections of either base layer can extend at a 90 degree angle. Alternatively, the microprojections of one base layer can extend at a 90 degree angle and the microprojections of a second base layer can extend at an angle less than 90 degrees such that the tips of the microprojections are closer than the bases of the microprojections.

In accordance with another aspect of the invention, a device for drug delivery includes a microprojection array with a plurality of stratum corneum piercing microprojections for piercing stratum corneum. At least some of the microprojections are from two different base layers and positioned in groups and in a group a continuous drug coating bridges the microprojections of the group.

In accordance with another aspect of the invention, a device for drug delivery includes a microprojection array with a plurality of stratum corneum piercing microprojections for piercing stratum corneum. At least some of the microprojections are from two different base layers and positioned in groups and where in a group at least one microprojection leans towards another microprojection and a continuous drug coating bridges the microprojections of the group.

In accordance with another aspect of the invention, a device for drug delivery includes a microprojection array with a plurality of stratum corneum piercing microprojections for piercing stratum corneum. At least some of the microprojections are from at least a first base layer and a second base layer and are positioned in groups. In at least some of the groups the microprojections have shafts of different lengths, a first microprojection extending normally from the microprojection member and a second microprojection leaning to the first microprojection forming a pinnacle. In at least some of the groups a continuous drug coating coats at least top portions of the microprojections in a group, the drug coating having a meniscus bridging the microprojections in the group.

In another aspect, the present invention further provides a method of making a device with microprojections, in any of the aspects described previously, to pierce stratum corneum to facilitate drug delivery by forming a multilayered microprojection member with at least a first base layer and second base layer, each with microprojections. Preferably a drug coating is coated on at least some of the microprojections. Various shapes and configurations, microprojection grouping, materials of construction and drug coating parameters can be selected to result in the desired designs of microprojection drug delivery devices.

In an aspect, the capability to stack layers of microprojections together enables an increase in the microprojection density with this invention, allowing a multifold increase in drug-coating. In a coating process, with the same number of passes, the drug loading can double if the number of microprojections is doubled.

Because the different microprojection layers can be formed separately, arrays of different designs and with different drug formulations can be combined as a single aligned register to create a single array with different types of microprojections. Thus, a single patch design can carry more than one type of therapeutic or biological compound and/or different dosages. Further, a single layer can have more than one type of therapeutic or biological compound and/or different dosages.

The ability to assemble together patterns with different microprojection designs can allow new features in the shaping of the microprojections to control skin penetration. For example, a design to limit skin penetration can be interwoven in-between every other microprojection such that the depth of skin penetration is controlled.

By stacking layers, new microprojection array designs can be made that facilitate better penetration of the microprojection through the stratum corneum and increase the drug loading with similar size of planar area in microprojection array. More drug can be loaded between adjacent microprojections and between base layers. This invention helps to increase the capacity of the microprojection to capture drug coating material on the microprojection, whereas otherwise a larger device with a larger volume and larger planar surface area would be required. This advantage, provided by increased drug loading without increasing planar area, is especially important for drugs that are less potent. Because large devices for piercing the stratum corneum are hard to handle and increase discomfort to the patient, the ability to increase drug loading on the device can be critical for patient compliance and the successful application of such a device. Coupling with the increased flexibility of loading a combination of drugs, the present invention provides substantial benefits for drug delivery not available in the past.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The present invention is illustrated by way of example in embodiments and not limitation in the figures of the accompanying drawings in which like references indicate similar elements. The figures are not shown to scale unless indicated otherwise in the content. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a sectional view of an applicator device and microprojection array system according to the present invention.

FIG. 2A illustrates an isometric view in portion of a microprojection array system according to the present invention.

FIG. 2B illustrates an exploded schematic view of two microprojection base layers with sections of microprojections having different drug coating formulations according to the present invention.

FIG. 3 illustrates a sectional view in portion of an embodiment of a pair of microprojections according to the present invention.

FIG. 4 illustrates an isometric view in portion of another embodiment of a pair of microprojections having a drug coating according to the present invention.

FIG. 5 illustrates an isometric view in portion of yet another embodiment of a group of microprojections forming a pinnacle according to the present invention.

FIG. 6 illustrates a sectional side view in portion of another embodiment of a group of microprojections according to the present invention.

FIG. 7 illustrates a sectional side view in portion of another embodiment of a group of microprojections according to the present invention.

FIG. 8 illustrates a sectional side view in portion of another embodiment of a group of microprojections with a drug coating with meniscus according to the present invention.

FIG. 9 illustrates an isometric view in portion of an embodiment of a group of microprojections showing microprojection layers according to the present invention.

FIG. 10 illustrates an isometric view in portion of yet another embodiment of a group of microprojections showing microprojection layers according to the present invention.

FIG. 11 illustrates an isometric in portion of yet another embodiment of a group of microprojections showing microprojection layers according to the present invention.

FIG. 12 shows a scanning electromicrograph of a double layered microprojection array having microprojection pairs.

FIG. 13 is a graph showing the drug content of a double layered microprojection member with paired microprojections compared to that of a single layered microprojection member without paired microprojections.

FIG. 14 showed the drug granisetron content of two layered microprojection members after a number of passes in dip coating.

FIG. 15 shows a scanning electromicrograph of another double layered microprojection array with drug coating.

FIG. 16A to FIG. 16C show the snap-fit for alignment of two layers.

FIG. 17A to FIG. 17C show the wedge-fit for alignment of two layers.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The present invention relates to methods and devices for transdermal delivery of drugs using a microprojection device in which a microprojection array arises from a microprojection member having at least two layers from which the microprojections arise. The multiple layered microprojection member increases the number of microprojection available and can allow more drug coating material to be held by the microprojections than single layered microprojection members.

In describing the present invention, the following terms will be employed, and are defined as indicated below. As used in this specification and the appended claims, the singular forms “a,” “an” and “the” include plural references unless the content clearly dictates otherwise.

As used herein, the term “transdermal” refers to the use of skin, mucosa, and/or other body surfaces as a portal for the administration of drugs by topical application of the drug thereto for passage into the systemic circulation. As described herein, the stratum corneum can be disrupted in such transdermal drug transport.

“Biologically active agent” is to be construed in its broadest sense to mean any material that is intended to produce some biological, beneficial, therapeutic, or other intended effect, such as enhancing permeation or relief of pain. As used herein, the term “drug” refers to any material that is intended to produce some biological, beneficial, therapeutic, or other intended effect, such as relief of pain, but not agents (such as permeation enhancers) the primary effect of which is to aid in the delivery of another biologically active agent such as the therapeutic agent transdermally.

As used herein, the term “therapeutically effective” refers to the amount of drug or the rate of drug administration needed to produce the desired therapeutic result.

The terms “microprojections” and “microprotrusions”, as used herein, refer to piercing elements that are adapted to pierce or cut through the stratum corneum into the underlying epidermis layer, or epidermis and dermis layers, of the skin of a living animal, particularly a mammal and more particularly a human.

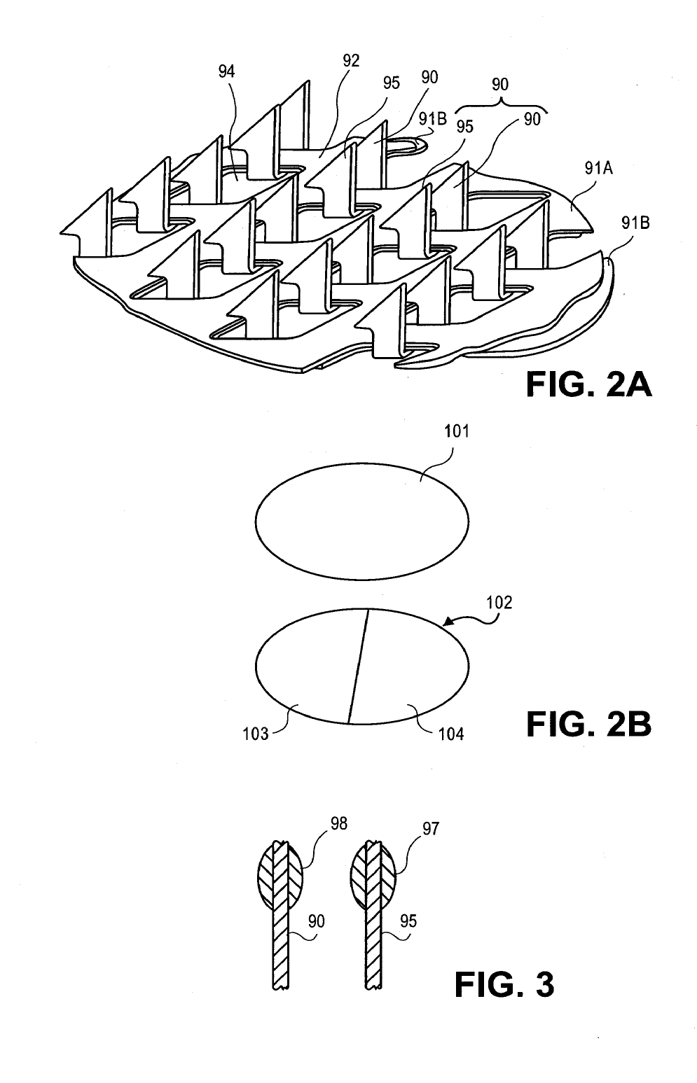

The term “microprojection array” or “microprotrusion array”, as used herein, refers to a plurality of microprojections arranged in an array for piercing the stratum corneum. The microprojection array may be formed by etching or punching a plurality of microprojections from a thin sheet or sheets and folding or bending the microprojections out of the plane of the sheet to form a configuration, such as the bent microprojections shown in FIG. 2A. Such methods of making microprojections are known in the art. For example, U.S. Pat. Nos. 5,879,326; 6,050,988; 6,091,975; 6,537,264 and US Patent Publication 20040094503 disclose processes for making microprojections by etching substrates. Silicon and plastic microprojection members are described in U.S. Pat. No. 5,879,326. The microprojection array can also be formed by other known methods, such as by forming one or more strips having microprojections along an edge of each of the strip(s) as disclosed in U.S. Pat. No. 6,050,988. These Patent publications are incorporated herein by reference in their entireties.

The term “group” when referred to microprojection arrangement means a plurality, e.g., two (a pair), or more, of neighboring microprojections that are closer to one another than to other microprojections. In many cases, there are repeating units of such groups of microprojections in the microprojection array.

The present invention involve devices and methodology that provide increased drug loading per unit size or planar surface area of a microprojection member having a microprojection array for piercing the stratum corneum. Through grouping microprojections in close proximity, such advantages over prior devices can be realized. For example, the microprojections in a group can have a continuous drug coating that bridges the microprojections.

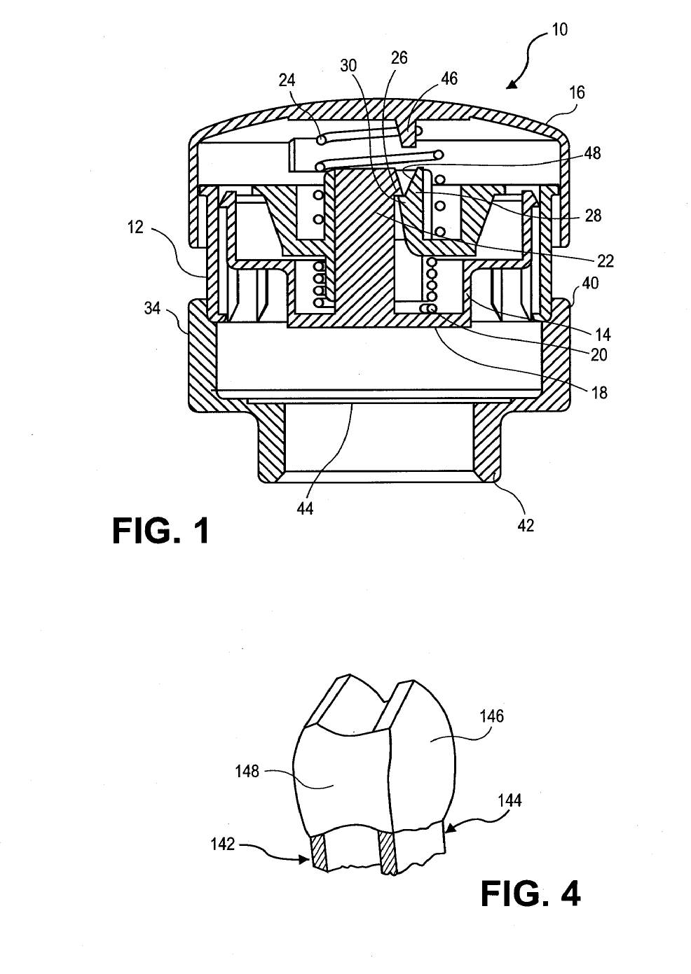

An applicator system for applying a microprojection member as described below includes an impact applicator for applying the microprojection member to the stratum corneum. The microprojection member can include a microprojection array. FIG. 1 shows a schematic sectional view of an exemplary microprojection device having an applicator, retainer, and microprojection array. Similar devices with actuators and retainers are described in United States patent documents 20020123675, 20050096586, 20050138926, 20050226922, and 20050089554, which are incorporated by reference herein. It is to be understood that such devices of these documents and other prior microprojection devices can be adapted to be used with the present invention. FIG. 1 illustrates an exemplary embodiment of an applicator 10 for use with a retainer 34 containing microprojection member 44. However, the device of FIG. 1 is just an example and other applicator configurations may also be used with the microprojection arrays described herein. The applicator 10 includes a body 12 and a piston 14 movable within the body. A cap 16 is provided on the body 12 for activating the applicator to impact the stratum corneum with the microprojection member 44. An impact spring 20 is positioned around a post 22 of the piston 14 and biases the piston downward (i.e., towards the skin) with respect to the body 12. The piston 14 has an impact surface 18 that is substantially planar, slightly convex, or configured to match the contours of a particular body surface. The surface 18 of the piston 14 impacts the microprojection member 44 against the skin causing the microprojections 90 to pierce the stratum corneum of, for example, the skin of a patient.

FIG. 1 shows the piston 14 in a cocked position. When the applicator is cocked, the piston 14 is pressed up inside the body 12 and locked in place by a locking mechanism. The locking mechanism includes a stop catch 26 on the post 22 and a flexible finger 28 on the body 12 having a corresponding latch stop 30. As the piston 14 is moved toward the body 12 compressing the impact spring 20, the stop catch 26 flexes the finger 28 and snaps over the corresponding latch stop 30 of the flexible finger. The cocking step is performed by a single compression motion that both cocks and locks the piston 14 in the cocked position.

In the cocked position, catch 26 and latch 30 on the piston 14 and body 12 are releasably engaged, preventing downward motion of the piston in the body. FIG. 1 also illustrates the patch retainer 34 mounted on the body 12. The activation of the applicator 10 by the release of the locking mechanism is performed by downward force applied to the applicator cap 16 while the end 42 of the applicator is held against the skin. The cap 16 is biased in a direction away from the skin by a hold down spring 24 that is positioned between the body 12 and the cap. The cap 16 includes a pin 46 extending downward from the cap. When the cap 16 is pressed downward against the bias of the hold down spring 24, the pin 46 contacts ramp 48 on flexible finger 28 moving the flexible finger outward and disengaging latch 30 of the flexible finger 28 from catch 26. This releases piston 14 and the piston moves downward impacting the stratum corneum with the microprojection member 44. The impact is applied substantially parallel to a central axis of the microprojection member 44. Preferably, the microprojection member is connected to the retainer by at least one frangible element (not shown in the figure) that is broken when the impact applicator is activated.

FIG. 2A illustrates an exemplary embodiment of a microprojection member having a microprojection array of the present invention. FIG. 2A shows a plurality of microprojections (or microprotrusions) in the form of microblades 90, which have a blade shape with a cutting sharp point. The microblades 90 extend at a substantially 90° angle from a sheet 92 having openings 94. The microprojections are preferably sized and shaped to penetrate the stratum corneum of the epidermis when pressure is applied to the microprojection member, for example, forming microslits on the body surface. The sheet 92 may be incorporated in an agent delivery patch or an agent-sampling patch that includes an agent (i.e., a pharmaceutical agent or drug) reservoir and/or an adhesive for attaching the patch to the stratum corneum.

It is preferred that at least some of the microprojections are arranged into groups. For example, in the embodiment shown in FIG. 2A, first microprojection 90 rising from first base layer 91A and second microprojection 95 arising from second base layer 91B are proximate to each other and form groups 96. In the group 96, microprojection 95 and microprojection 96 are closer to one another than to other microprojections that are not in the group. Base layer 91A has window openings 94 to allow second microprojections 95 to protrude through to pair with the first microprojection 90. It is desirable, but not necessary, that all the microprojections of one base layer are paired or matched with microprojections from another layer. Some microprojections can remain ungrouped. Preferably a number of such groups are present as repeated units in the microprojection array.

Preferably the microprojections each have a drug coating with a drug (for example, on or near the tip of the microprojections). The microprojection member and microprojection array can be made with technology known in the art. Examples of agent delivery and sampling patches that incorporate a microprojection array are found in US20020016562, U.S. Pat. No. 6,537,264, WO 97/48440, WO 97/48441, WO 97/48442, the disclosures of which are incorporated herein by reference in their entireties. The microprojection array of FIG. 2A without a drug reservoir or a drug coating may also be applied alone as a skin pretreatment. In one embodiment of the invention, the microprojections have projection length of less than 1000 microns (μ). In a further embodiment, the microprojections have a projection length of less than 500 microns (μ), more preferably, less than about 250μ. In some embodiments, the microprojections preferably have a normally extending portion of about 25μ to 400μ long, more preferably about 50μ to 250μ long. As used herein, “normally extending” means extending at an angle from the plane of a microprojection member and, although possible, need not be exactly 90°.

Because microprojections are small and are often made from a flat sheet of material, there is usually a sizable gap between adjacent microprojections made from the same sheet. Stacking multiple layers of base layers each having microprojections allows microprojections from one base layer to be inserted in the gaps between microprojections from the other base layer. In this way, more microprojections can be placed in a unit planar area. For example, if a base layer has 500 microprojections/cm², then stacking two base layers together with the microprojections of one base layer matching microprojections of the other layer will about double the number of microprojections per unit planar area. This is particularly beneficial for drugs that are less potent and would otherwise not be able to deliver the effective dose for desired biological effect. By increasing the number of microprojections per unit planar area in a microprojection member, the capacity for loading drug is increased. As used herein, unless specified to be otherwise, “planar area” of a microprojection member refers to the overall area of the microprojection member without subtracting off the area of the openings. However, even with the area of the openings being accounted for, if base layers are stacked so that openings of different layers overlap, drug loading capability per unit exposed area for the microprojection is increased with the present invention compared to prior devices.

Another advantage of stacking base layers together is that this configuration allows the delivery of different (i.e. multiple) drugs simultaneously. For example, a first drug can be loaded by means of a first drug coating on the microprojection of a first base layer. A second drug can be loaded by means of a second drug coating on the microprojection of a second base layer. The two layers can then be stacked. Such a system will be particularly beneficial for two drugs that require different drug coating formulation to incorporate the desired drug loading for therapy. For example, the two drugs may have different solubility in different solvents, thereby requiring different formulations. Also, as an example, one microprojection can be coated with a formulation that exhibits a fast therapeutic onset, while the other registered can be coated with a formulation that exhibits a sustained therapeutic effect. Further, the microprojections can have different dosage. The two layers can be made separately with different solvents, polymers, thickeners, etc., to optimize the chemical and physical parameters in the respective formulations on the respective drugs.

Yet another advantage of stacking base layers is that one layer can have different sections (areas) with different drugs, and the microprojections of the base layers can match together. This way, more than two drugs (also, more than three drugs, etc.) can be delivered simultaneously. For example, as shown schematically in an exploded view in the embodiment of FIG. 2B, a first layer 101 can have a single drug (first drug) and the second layer 102 can have a section 103 with a second drug and another section 104 with a third drug. For example, half of the planar surface of the second layer can have the second drug and the other half can have the third drug. The two sections 103, 104 of the second layer 102 can be rigidly affixed to the first layer 101 so all of them are rigidly held together as a unit. As used herein, “rigidly held together” means that the relative position of the microprojections of the two layers are maintained although the baselayers may still be slightly flexible as a whole. Obviously, any layer can have multiple sections and this methodology can be extended to more layers, more sections, with more drugs.

Because the microprojections of one layer can protrude through openings of the other layer, microprojections of the two layers can be placed close together forming groups. One of the advantages of grouping microprojections together is that they can penetrate the stratum corneum easier. Since skin is supple and flexible, when a pointed object is pressed onto the skin, it pushes the skin inward but does not immediately penetrate. This is analogous to when a pencil tip is gently pushed against the skin the pencil will cause the skin surface to dimple but the skin does not allow the pencil point to break through the skin surface. Having a group (e.g., two) of microprojections close together will allow the skin to be taut between the microprojections in the group when the microprojections are pressed against the skin and therefore allow easier penetration. This will be particularly useful if the microprojections are relatively short and may not be able to penetrate adequately otherwise.

As mentioned before, microprojections can have a drug coating to carry the drug to be delivered and stacking base layers with microprojections can increase the number of microprojections per unit area, thereby increasing the drug loading. FIG. 3 shows an embodiment of a group (e.g., a pair) of neighboring microprojections 90, 95 having drug coatings 97, 98 at the distal portions (or top portions) thereof. As used herein, “distal” means a direction that is towards the skin surface on which the microprojection is to be applied. In FIG. 3 the microprojections are substantially parallel to each other and the drug coatings 97, 98 from the two microprojections 95, 96 do not touch.

A further way to increase drug loading is to group neighboring microprojections close enough together to capture a continuous drug coating between the microprojections in the group. Thus, arranging the microprojections into a group will increase the volume of drug coating that can be held than otherwise possible. FIG. 4 illustrates an embodiment of a group (which in this case is a pair) of microprojections 142, 144. The microprojections 142, 144 extend in an about parallel fashion. A continuous drug coating 146 coats and extends from one microprojection 142 near its top to the other microprojection 144, forming a drug coating bridge 148. Thus, drug coating material bridges the microprojection 142, 144 and is sandwiched therebetween. Also, the drug coating material of the drug coating bridge 148 actually is continuous over and envelops the top portion of the microprojections 142, 144.

The grouping of microprojections in close proximity allows the adjacent microprojections to act as a “parallel plane capillary” and to shield and protected the drug coating therebetween from the impact forces during skin penetration, allowing the drug coating to penetrate deeper into the skin for effective drug delivery without coming off by the impact. In certain groups enabled by the present invention, adjacent microprojections converge in a way that skin penetration is facilitated.

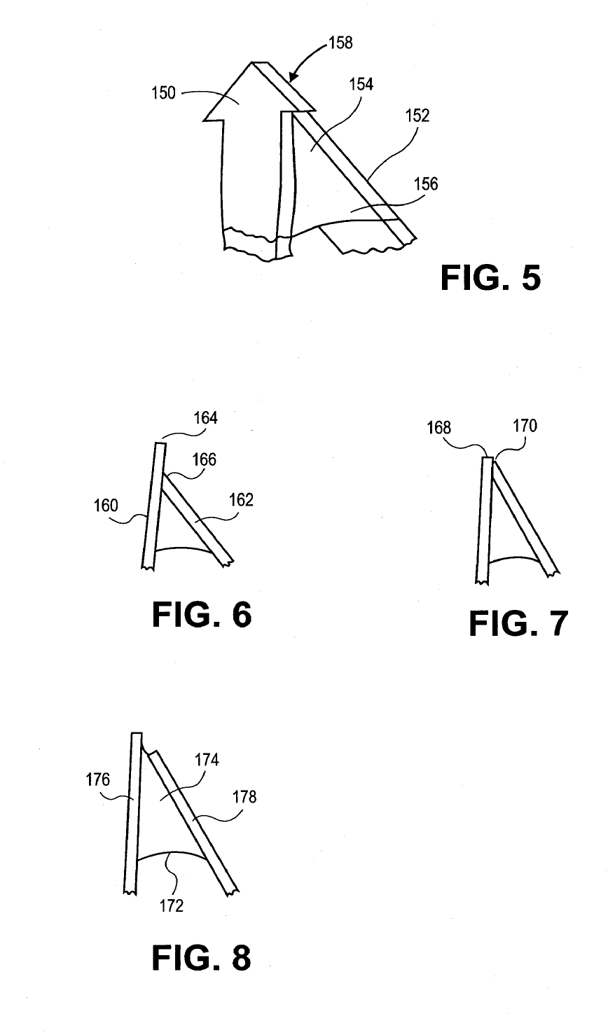

FIG. 5 shows an illustration of another alternative with a group (here a pair) of microprojections converging at the tips. In the embodiment of FIG. 5, microprojection 150 extends substantially straight up from the microprojection member planar plate (not shown) and microprojection 152 leans at an angle toward microprojection 150 so that the drug coating 154 forms a continuous bridge 156 coating the top portions of both of the microprojections. In this embodiment, microprojection 150 has an arrowhead shaped top portion. The converging of microblades forms a pinnacle 158 that can facilitate penetration of the stratum corneum. The angle of leaning (relative to the plane of the microprojection member) preferably is about 60° to slightly less than 90°, more preferably about 70° to 80°. The leaning microprojection can be longer, the same length or shorter than the one that is not leaning. Furthermore, one, two or more of the microblades in the group can be leaning.

The microblades can converge such that their tips are close together but not exactly touching. Alternatively, the microblades can converge to touch at the tips. Further, as shown in FIG. 6, one microblade (say, a first microblade) 160 can intercept a second microblade 162 along by the elongated portion of the first microblade 160 such that tip 164 of the first microblade 160 extends past the tip 166 and the body of the second microblade 162 (but not the other way around). The tip 166 of the second microprojection 162, although touching the first microprojection 160 in this embodiment, does not extend past the first microprojection. This way, during penetration of the stratum corneum, the tip 164 of first microblade 160 will initiate the penetration. Alternatively, the microblades can converge such that their tips 168, 170 are about even, as shown in FIG. 7. This way, the tips 168, 170 of the microblades generally penetrate the stratum corneum at about the same time.

The proximity of microprojections in a group allows the drug coating liquid before solidifying to be drawn and held by capillary action among the microprojections in a group. This is especially useful in embodiments with converging top portions because the capillary action tends to draw the liquid drug coating towards the tips of the microprojections, and therefore at a position suitable to delivery drug deeper into the skin. This phenomenon is especially evident in instances in which hydrophilic drug coating composition is coating hydrophilic microprojections, wherein there is a small contact angle for the liquid on a surface. Wetability of a liquid on a surface is related to the contact angle θ formed by the liquid-solid and the liquid-gas interfaces. If θ is greater than 90° the liquid tends to form droplets on the surface, i.e., the liquid does not wet the surface well. If θ is less than 90° the liquid tends to spread out over the surface. When the liquid forms a thin film on the surface, i.e., wetting it well, θ tends to near zero. In instances of hydrophilic liquid on a hydrophilic surface, for example, as shown in FIG. 8, a concave shaped meniscus 172 would be formed by the capillary force in the drug coating 174 on the top portion of microprojections 176, 178 in a group. As used herein, even after the drug coating has solidified, the concaved shaped curve 172 is still called a meniscus for the sake of consistency. In FIG. 8, the tips of the microprojections 176 178 do not actually touch. However, the drug coating 172, due to its viscosity before solidifying, still envelops the top portions of the microprojections and forms a bridge of continuous drug coating material between them. The bulk of the drug coating material is held between the microprojections in this embodiment.

The convergence of the top portions of the microprojections in a group further functions to protect the drug coating from being pushed off the top portions of the microprojections because much of the drug coating is, for example, under the pinnacle formed by the tips of the microprojections and therefore shielded by the tips of the microprojections during penetration of the stratum corneum. In an embodiment in which the top portions of microprojections in a group are apart sufficiently on top at the tips as well as lower in the shafts of the microprojections, there can be a meniscus on the top of the drug coating as well as in the bottom of the drug coating, similar to what is shown in FIG. 4.

A microprojection array can be made, for example, from a sheet of material by chemical etching. Methods for forming structures that are small (in the range of tens to hundreds of microns) by chemical etching are known in the art. A substrate material, generally flat as a sheet, such as a titanium sheet, can be chemically etched. In generally, a photoresist or a photo-sensitive polymer is laid on a substrate. A pattern is imaged on the photoresist (e.g., with ultra-violet light) and then the photoresist is then developed to provide a patterned polymer layer on the substrate. The patterned polymer layer protects portions of the substrate and leaves other portions unprotected. The substrate with the patterned polymer layer is exposed to an etching liquid, for example, as in a process of spraying the etching liquid on the substrate (with the patterned polymer layer thereon). The part of the substrate that is not protected by the patterned polymer layer is corroded, forming a patterned substrate having microblades that lie flat along the plane of the substrate. The microblades are then cleaned.

The microblades are bent using dies. A microblade is bent such that an elongated portion extends normally from the plane of the substrate. When a microprojection array is made this way, the resulting microprojection array on a microprojection member has the microblades, including the top portions and the bottom portions, and the rest of the base layer are made of the same continuous piece material and is an integral piece.

Depending on whether the microprojections of the different layers are to be coated with the same drug, same coating material, or different drug or different coating material, the microprojections can be stacked before coating or coated before stacking. After stacking, the base layers can be rigidly affixed together by methods known in the art, such as thermal joining, using adhesive, and the like. Thermal joining can be done, e.g., by thermal fusion achieved through high temperature and pressure (diffusion bonding), or localized metal fusion (such as with high current welding or heat). Gas shielding (nitrogen or argon, for example) can be used during assembly to ensure pure substrate (e.g., titanium) chemical composition and improve results of the heated areas. Means, such as heat sink, can be used for protecting the drug coating if the layers are thermally joined after drug coating is done.

As mentioned, microprojection array with groups of microprojections can be made, for example, by stacking two layers of microprojections together so the microprojections of one layer protrudes through openings of the other layer. There are many ways to stack layers of microprojections together. One embodiment has been shown in FIG. 2A, in which a microprojection 95 of the bottom base layer 91B (bottom as seen in the figure) is separated from its matched microprojection 90 of the top base layer 91A by a planar portion of the top base layer, which is between the two microprojections.

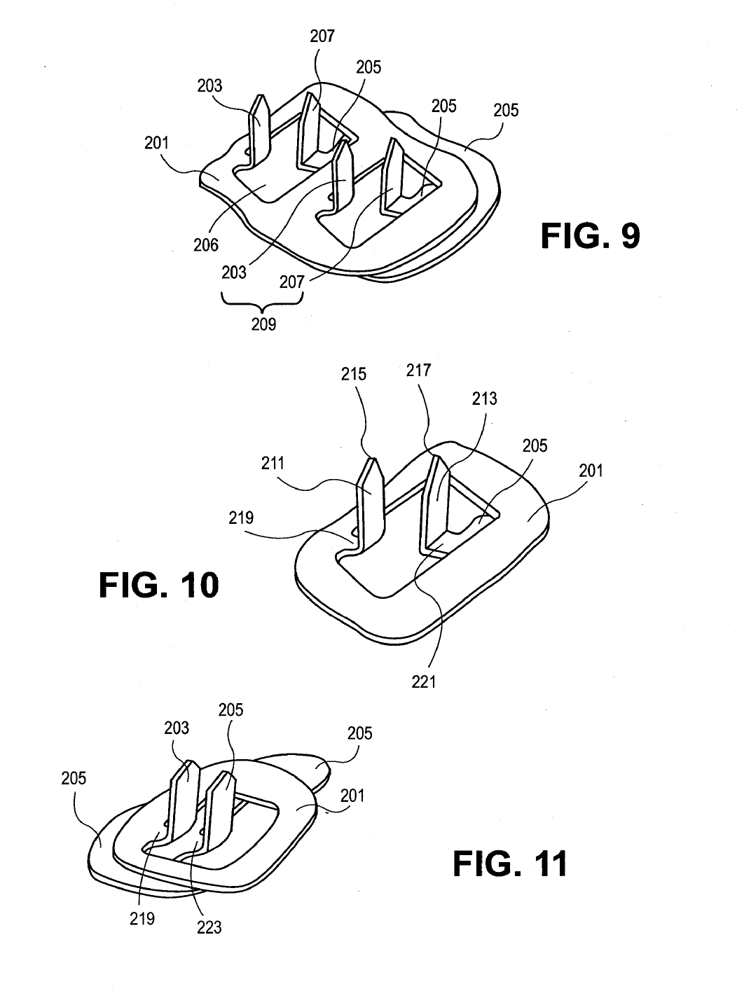

Another embodiment is shown in FIG. 9. As shown in FIG. 9, a top microprojection base layer (or simply “top microprojection layer”) 201 has top microprojections 203 extending out of the plane of the top microprojection layer 201. On the top microprojection layer 201 are a plurality of top openings 206. The top microprojection 203 is positioned near the edge of the top openings 206. A bottom microprojection base layer (or simply “bottom microprojection layer”) 205 is situated under the top microprojection layer 201. A plurality of bottom microprojections 207 arising from the bottom microprojection layer 201 extend through the top openings 206 near the top microprojections 203 to form groups 209 of microprojections.

FIG. 10 shows the embodiment of FIG. 9 in more detail. In the embodiment of FIG. 9 and FIG. 10, the microprojections in the top microprojection layer 201 have a shorter distally (i.e., upwardly in the figure) extending top portion 211 than the distally extending top portions 213 of the bottom microprojection layer 205. In this way, the tips 215 of the top microprojections 211 and the tips 217 of the bottom microprojections are about even over the whole microprojection member, which is composed of the top microprojection layer 201 and the bottom microprojection layer 205, including the corresponding microprojections thereon. Alternatively, the distally extending top portions of the top microprojections can have about the same length as the upwardly extending top portions of the bottom microprojections. To prevent relative movement between the top microprojections and the bottom microprojections, the two microprojection layers 201, 205 can be thermally joined together, e.g., by welding or other techniques known in the art. When stacked together so that their microprojections together form a microprojection array, the two or more microprojection layers can be considered as a single microprojection member.

In the embodiment shown in FIG. 9 and FIG. 10, in a group of microprojections, a planar portion (extending along the plane of the base layer, e.g., 219, 221) of microprojection (e.g., 203, 207) from each microprojection layer (e.g., top layer and bottom layer) points toward the microprojection (e.g., 207, 203) of the other layer. The planar portions and the top portions of the microprojections were formed by bending or lifting the top portion of the microprojections from the plane of the sheet material after etching. Of course, another alternative, as shown in FIG. 11, is to have the two microprojection layers 201, 205 stacked together such that in a group one planar portion 219 of microprojection of a first layer 201 points toward a planar portion 223 of microprojection of a second layer 205 while the microprojection planar portion 223 from the second layer 205 points away from the microprojection planar portion 219 of the first layer 201.

One of the advantages of the configurations of FIGS. 9, 10, and 11 is that the microprojections can be placed very close together because there is no base layer material interposing between the microprojections in a group like that shown in FIG. 2A. Further, configurations of FIGS. 9 and 10 can be designed such that the windows of the two layers can match so that the edges of the two layers are flush. The reason is that the planar portions of the microprojections allow the microprojections to be within the perimeter of the windows thereby allowing the edges of the two layers (and if desired, the edges of the windows) to match flush. The matching of the edges of the two layers (or more) allows the layers to be efficiently affixed together. An advantage of the design of FIG. 11 is that the two layers can have the same basic design and they can be stacked well together. Further, when openings of the two base layers are matched, there is a continuous space in the openings void of base layer materials. This void space can be filled or partially filled with drug coating material.

To further increase drug loading, one or more depressions can be formed on the surface of the face of the microblades. The depressions can have a variety of shapes, such as round, oval, polygonal, elongated, star-shaped, and the like. A preferred shape is an elongated channel formed along the shaft of the microprojection, e.g., along the top portion of the microprojection. Further, the microprojection can have a depression on each of the two faces of the microblade. The depressions can extend through the microblade forming a throughhole. The depression can be on a face of the microprojection facing the other microprojection in the group or it can be on the face facing away from the microprojection in the group. In some embodiments, depressions can be located on one microprojection or on multiple microprojections in the group. Thus, the microprojections can increase the drug loading by providing more surface area on the microprojections and providing a large volume between the microprojections.

In another alternative one face of a microblade can be sculptured to have a depression, such as a channel, and the other face can have a more rounded, or bowed surface akin to a portion of an annular convex surface. For example, the microblade can have an elongated channel on one face and a bowed elongated back on the opposite face. In this way, the microblade has a top portion that is generally thumbnail shape.

The top portion, including the tip, of a microprojection can also have a variety of shapes. For example, the top portion can have an arrowhead shape (e.g., as shown in FIG. 5), a half-arrowhead shape (like that shown in FIG. 2A), a tombstone shape with a wedge-shaped top (as shown in FIG. 4), a rounded top, a flat top, and the like.

The microprojections can be formed from metallic materials such as titanium, stainless steel, and polymers. Techniques for making microprojection array (e.g., by etching) from such materials are known in the art. Generally, substrates for forming microprojections are about 3 microns (μm) to 50 μm thick, preferably about 15 μm to 35 μm thick. The microprojections typically have a width of about 5 μm to 250 μm, preferably about 100 μm to 150 μm. The thicknesses of the microprojections are about 3 μm to 50 μm, preferably about 10 μm to 30 μm. The microprojections may be formed in different shapes, such as needles, blades, pins, punches, and combinations thereof. If the microprojections are from the same sheet of material (for example, all were chemically etched from the same single sheet of titanium), the microprojection density is approximately 10 microprojections/cm², more preferably, in the range of approximately 200-5000 microprojections/cm². The distance between neighboring microprojections in a group can be about less than about 500 μm, preferably less than about 200 μm, more preferably about 10 μm to 160 μm, even more preferably about 10 μm to 100 μm, even more preferably about 50 μm to 100 μm, at the base of the microprojections. Typically the microprojections extend from a base plate upward. The distances are generally measured between the base positions of the upwardly extending portions. There can be openings near the microprojections on the microprojection member. Such openings can allow agents or drugs to pass if agents or drugs are placed under or in such openings. Drug can also be held between two base layers, for example, as a drug coating composition that is placed between two base layers through the openings by capillary force and dried. The number of openings per unit area through which the active agent (drug) passes is preferably from approximately 10 openings/cm² to about 2000 openings/cm².

After two or more microprojection layers are forms, the two or more layers can be aligned and affixed together. For example, the edges of the openings or windows or of the layers can be aligned, or alignment means such as aligning projections and receptors for these aligning projections between layers can be used. There are various ways to affix the layer together. One way is by thermal fusion (welding). Because precise alignment of the layers to match the microprojections and openings is important, a good and precise way to align the layers is beneficial. One way to help alignment is to form a layer with latches (aligning projections) that can fit into catches (receptors for the aligning projections) of another layer. With multiple latches on one layer and multiple catches in another layer, when the latches are frictionally fit (or interference fit) together, the two layers (or even more layers) are aligned. After the layers are frictionally (or interference) fitted together, they can be permanently affixed together, e.g., by thermal fusion.

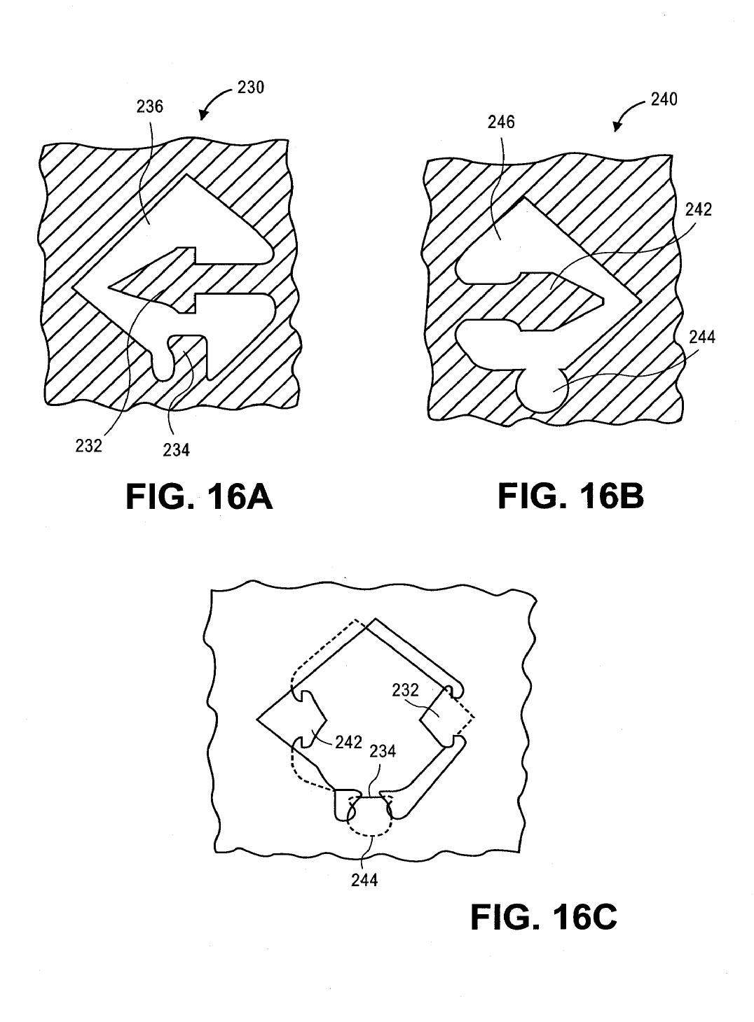

For example, FIG. 16A shows the design of a microprojection cell 230 with a microblade 232, a latch 234 and an opening 236 for a top microprojection layer. FIG. 16B shows the design of a microprojection cell 240 with a microblade 242, a catch 244 and an opening 246 for a bottom microprojection layer. After the cells are formed by chemically etching substrates, the microblades are lifted to extend from the planes of the substrates. Further the latches are also made to angle from the plane of the substrate such that when the two microprojection layers are aligned and pressed together, the latch 234 from the top microprojection layer is pressed and frictionally fit into the catch 244 of the bottom microprojection layer, as shown in FIG. 16C, in which the dotted line shows portions of the outline of the features that are hidden from view. The latches can fit into the catches with a snap or click as the layers are pressed together. Thus, this type of frictional fit can be called “click fit” or “snap fit”. Of course, although snapping movement and clicking sound are possible, the latches and the catches can be designed in a way that when the layers are pressed together at least some of the latches and some of the catches are frictionally fit (or interference fit) and hold together and the fitting together of the layer not necessarily resulting in a snapping movement or clicking sound.

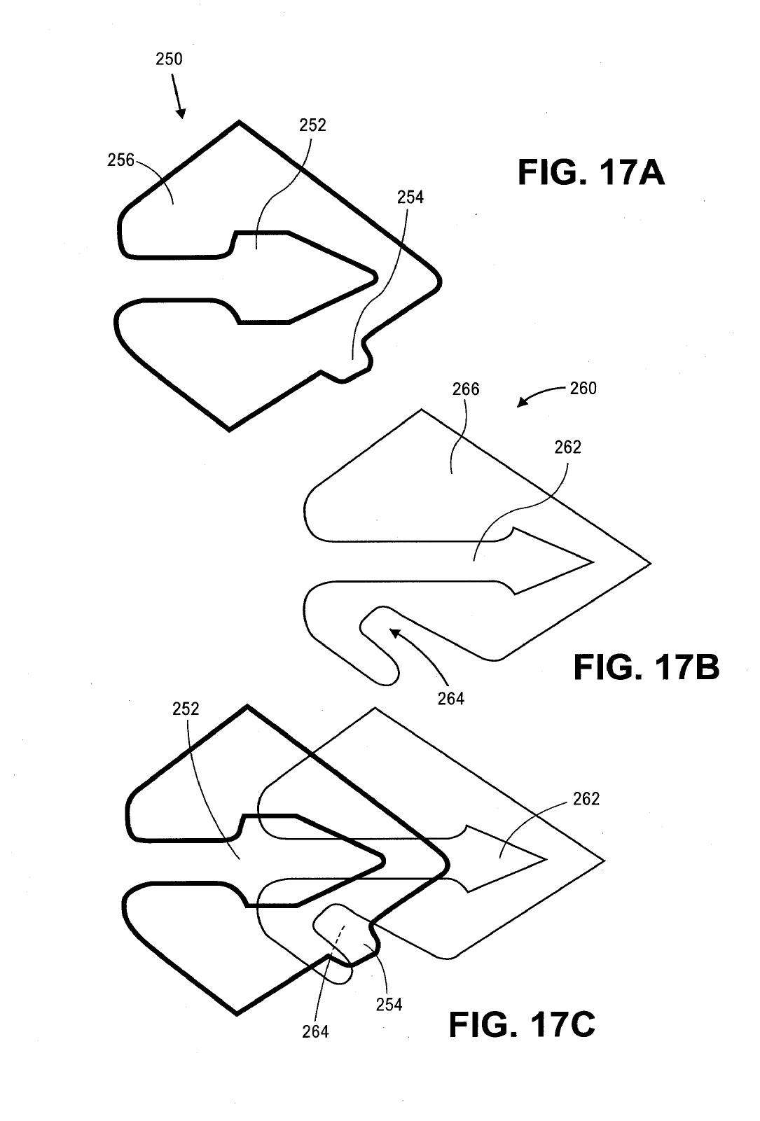

FIG. 17A to FIG. 17C are schematic views showing another embodiment. FIG. 17A shows a microprojection cell 250 with a microblade 252, a latch 254 and an opening 256 for a top microprojection layer. FIG. 17B shows the design of a microprojection cell 260 with a microblade 262, a catch 264 and an opening 266 for a bottom microprojection layer. After the cells are formed by etching from substrates, the latch 254 is bent to angle from the plane of the substrate of the top microprojection layer suitable for wedging into the catch 264 of the bottom microprojection layer. The two layers can be pressed to wedge the latches from one layer into the catches of the other layer. FIG. 17C is a schematic drawing showing the wedging relationship with frictional fit (or interference fit) between the latch 254 and the catch 264 but the microprojections 252 and 262 are not shown to have been bent, for better illustrating the positions of the cells 250, 260. It is noted that layer can have latches alone, catches alone or a combination of latches and catches so long as they can match and fit with corresponding catches and/or latches of another layer. Again, it is not necessary that there be any sudden snapping movement or sound and it is not necessary that all cells have either a latch or a catch so long as there are enough latches and catches in the layers to align the layers well.

The drug coating can include one or more of a variety of drugs or biologically active agents. Such drugs include traditional pharmaceuticals, as well as small molecules and biologics. Examples of such drugs or biologically active agents include, without limitation, leutinizing hormone releasing hormone (LHRH), LHRH analogs (such as goserelin, leuprolide, buserelin, triptorelin, gonadorelin, and napfarelin, menotropins (urofollitropin (FSH) and LH)), vasopressin, desmopressin, corticotrophin (ACTH), ACTH analogs such as ACTH (1-24), calcitonin, vasopressin, deamino[Val4, D-Arg8] arginine vasopressin, interferon alpha, interferon beta, interferon gamma, erythropoietin (EPO), granulocyte macrophage colony stimulating factor (GM-CSF), granulocyte colony stimulating factor (G-CSF), interleukin-10 (IL-10), glucagon, growth hormone releasing factor (GHRF), insulin, insulinotropin, calcitonin, octreotide, endorphin, TRN, NT-36 (chemical name: N[[(s)-4-oxo-2-azetidinyl]carbonyl]-L-histidyl-L-prolinamide), liprecin, aANF, bMSH, somatostatin, bradykinin, somatotropin, platelet-derived growth factor releasing factor, chymopapain, cholecystokinin, chorionic gonadotropin, epoprostenol (platelet aggregation inhibitor), glucagon, hirulog, interferons, interleukins, menotropins (urofollitropin (FSH) and LH), oxytocin, streptokinase, tissue plasminogen activator, urokinase, ANP, ANP clearance inhibitors, BNP, VEGF, angiotensin II antagonists, antidiuretic hormone agonists, bradykinin antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, prostaglandin antagonists, pentigetide, protein C, protein S, renin inhibitors, thymosin alpha-1, thrombolytics, TNF, vasopressin antagonists analogs, alpha-i antitrypsin (recombinant), TGF-beta, fondaparinux, ardeparin, dalteparin, defibrotide, enoxaparin, hirudin, nadroparin, reviparin, tinzaparin, pentosan polysulfate, oligonucleotides and oligonucleotide derivatives such as formivirsen, alendronic acid, clodronic acid, etidronic acid, ibandronic acid, incadronic acid, pamidronic acid, risedronic acid, tiludronic acid, zoledronic acid, argatroban, RWJ 445167, RWJ-671818, fentanyl, remifentanyl, sufentanyl, alfentanyl, lofentanyl, carfentanyl, and mixtures thereof.

The drugs or biologically active agents can also be in various forms, such as free bases, acids, charged or uncharged molecules, components of molecular complexes or nonirritating, pharmacologically acceptable salts. Further, simple derivatives of the active agents (such as ethers, esters, amides, etc.), which are easily hydrolyzed at body pH, enzymes, etc., can be employed.

The drugs or biologically active agents can be incorporated into a liquid drug coating material and coated onto the microprojections.

Typically, the drug or biologically active agent is present in the drug coating formulation at a concentration in the range of approximately 0.1-30 wt %, preferably 1-30 wt %.

Preferably, the amount of drug contained in the biocompatible coating (i.e., dose) is in the range of approximately 1 μg-1000 μg, more preferably, in the range of approximately 10-200 μg per dosage unit. Even more preferably, the amount of the drug contained in the biocompatible coating is in the range of approximately 10-100 μg per dosage unit.

Preferably, the pH of the coating formulation is adjusted to provide conditions for maintaining the stability of the drug selected for incorporation in the drug coating formulation. In certain embodiments of the invention, the viscosity of the coating formulation is enhanced by adding low volatility counterions. In certain embodiments, the drug has a positive charge at the formulation pH and the viscosity-enhancing counterion comprises an acid having at least two acidic pKas. Suitable acids include, without limitation, maleic acid, malic acid, malonic acid, tartaric acid, adipic acid, citraconic acid, fumaric acid, glutaric acid, itaconic acid, meglutol, mesaconic acid, succinic acid, citramalic acid, tartronic acid, citric acid, tricarballylic acid, ethylenediaminetetraacetic acid, aspartic acid, glutamic acid, carbonic acid, sulfuric acid and phosphoric acid.

In the noted embodiments of the invention, the amount of counterion is preferably sufficient to neutralize the charge of the drug. In such embodiments, the counterion or the mixture of counterion is preferably sufficient to neutralize the charge present on the agent at the pH of the formulation. In additional embodiments, excess counterion (as the free acid or as a salt) is added to the drug to control pH and provide adequate buffering capacity.

In one embodiment, the counterion comprises a viscosity-enhancing mixture of counterions chosen from the group consisting of citric acid, tartaric acid, malic acid, hydrochloric acid, glycolic acid and acetic acid. Preferably, the counterions are added to the formulation to achieve desired viscosity.

The viscosity of the drug coating formulation in liquid form is affected by the nature of the polymeric material and counterions present. The drug coating formulations typically have a viscosity of less than approximately 500 centipoise (typically measured at 25° C. and at a shear strain rate of 100/sec) and greater than 3 centipoise (cp), preferably a viscosity in the range of about 20-200 cp. Such viscosity ranges are suitable for forming a drug coating on the microprojections, for example, wherein capillary force can hold the liquid drug coating formation between the microprojections in a group until the formulation is solidified.

In certain embodiments, the viscosity-enhancing counterion contains an acidic counterion, such as a low volatility weak acid. Preferably, the low volatility weak acid counterion exhibits at least one acidic pKa and a melting point higher than about 50° C. or a boiling point higher than about 170° C. at atmospheric pressure. Examples of such acids include, without limitation, citric acid, succinic acid, glycolic acid, gluconic acid, glucuronic acid, lactic acid, malic acid, pyruvic acid, tartaric acid, tartronic acid and fumaric acid.

In another embodiment, the counterion comprises a strong acid. Preferably, the strong acid exhibits at least one pKa lower than about 2. Examples of such acids include, without limitation, hydrochloric acid, hydrobromic acid, nitric acid, sulfonic acid, sulfuric acid, maleic acid, phosphoric acid, benzene sulfonic acid and methane sulfonic acid. Another embodiment is directed to a mixture of counterions, wherein at least one of the counterion comprises a strong acid and at least one of the counterions comprises a low volatility weak acid.

Another preferred embodiment is directed to a mixture of counterions, wherein at least one of the counterions comprises a strong acid and at least one of the counterions comprises a weak acid with high volatility. Preferably, the volatile weak acid counterion exhibits at least one pKa higher than about 2 and a melting point lower than about 50° C. or a boiling point lower than about 170° C. at atmospheric pressure. Examples of such acids include, without limitation, acetic acid, propionic acid, pentanoic acid and the like.

The acidic counterion is preferably present in an amount sufficient to neutralize the positive charge present on the drug at the pH of the formulation. In additional embodiments, excess counterion (as the free acid or as a salt) is added to control pH and to provide adequate buffering capacity.

In another embodiment of the invention, the coating formulation includes at least one buffer. Examples of such buffers include, without limitation, ascorbic acid, citric acid, succinic acid, glycolic acid, gluconic acid, glucuronic acid, lactic acid, malic acid, pyruvic acid, tartaric acid, tartronic acid, fumaric acid, maleic acid, phosphoric acid, tricarballylic acid, malonic acid, adipic acid, citraconic acid, glutaratic acid, itaconic acid, mesaconic acid, citramalic acid, dimethylolpropionic acid, tiglic acid, glyceric acid, methacrylic acid, isocrotonic acid, β-hydroxybutyric acid, crotonic acid, angelic acid, hydracrylic acid, aspartic acid, glutamic acid, glycine and mixtures thereof.

In one embodiment of the invention, the coating formulation includes at least one antioxidant, which can be sequestering agents, such sodium citrate, citric acid, EDTA (ethylene-dinitrilo-tetraacetic acid) or free radical scavengers such as ascorbic acid, methionine, sodium ascorbate and the like. Presently preferred antioxidants comprise EDTA and methionine.

In the noted embodiments of the invention, the concentration of the antioxidant is in the range of approximately 0.01-20 wt. % of the coating formulation. Preferably the antioxidant is in the range of approximately 0.03-10 wt. % of the coating formulation.

In one embodiment of the invention, the coating formulation includes at least one surfactant, which can be zwitterionic, amphoteric, cationic, anionic, or nonionic, including, without limitation, sodium lauroamphoacetate, sodium dodecyl sulfate (SDS), cetylpyridinium chloride (CPC), dodecyltrimethyl ammonium chloride (TMAC), benzalkonium, chloride, polysorbates, such as Tween 20 and Tween 80, other sorbitan derivatives, such as sorbitan laurate, alkoxylated alcohols, such as laureth-4 and polyoxyethylene castor oil derivatives, such as CREMOPHOR EL.

In one embodiment of the invention, the concentration of the surfactant is in the range of approximately 0.01-20 wt % of the coating formulation. Preferably the surfactant is in the range of approximately 0.05-1 wt % of the coating formulation.

In a further embodiment of the invention, the coating formulation includes at least one polymeric material or polymer that has amphiphilic properties, which can comprise, without limitation, cellulose derivatives, such as hydroxyethylcellulose (HEC), hydroxypropylmethylcell-ulose (HPMC), hydroxypropycellulose (HPC), methylcellulose (MC), hydroxyethylmethylcellulose (HEMC), or ethylhydroxy-ethylcellulose (EHEC), as well as pluronics.

In one embodiment of the invention, the concentration of the polymer presenting amphiphilic properties in the coating formulation is preferably in the range of approximately 0.01-20 wt %, more preferably, in the range of approximately 0.03-10 wt. % of the coating formulation.

In another embodiment, the coating formulation includes a hydrophilic polymer selected from the following group: hydroxyethyl starch, carboxymethyl cellulose and salts of, dextran, poly(vinyl alcohol), poly(ethylene oxide), poly(2-hydroxyethylmethacrylate), poly(n-vinyl pyrolidone), polyethylene glycol and mixtures thereof, and like polymers.

In a preferred embodiment, the concentration of the hydrophilic polymer in the coating formulation is in the range of approximately 1-30 wt %, more preferably, in the range of approximately 1-20 wt % of the coating formulation.

In another embodiment of the invention, the coating formulation includes a biocompatible carrier, which can comprise, without limitation, human albumin, bioengineered human albumin, polyglutamic acid, polyaspartic acid, polyhistidine, pentosan polysulfate, polyamino acids, sucrose, trehalose, melezitose, raffinose, stachyose, mannitol, and other sugar alcohols.

Preferably, the concentration of the biocompatible carrier in the coating formulation is in the range of approximately 2-70 wt %, more preferably, in the range of approximately 5-50 wt % of the coating formulation.

In another embodiment, the coating formulation includes a stabilizing agent, which can comprise, without limitation, a non-reducing sugar, a polysaccharide or a reducing sugar.

Suitable non-reducing sugars for use in the methods and compositions of the invention include, for example, sucrose, trehalose, stachyose, or raffinose.

Suitable polysaccharides for use in the methods and compositions of the invention include, for example, dextran, soluble starch, dextrin, and insulin.

Suitable reducing sugars for use in the methods and compositions of the invention include, for example, monosaccharides such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as, for example, primeverose, vicianose, rutinose, scillabiose, cellobiose, gentiobiose, lactose, lactulose, maltose, melibiose, sophorose, and turanose, and the like.

Preferably, the concentration of the stabilizing agent in the coating formulation is at ratio of approximately 0.1-2.0:1 with respect to the drug, more preferably, approximately 0.25-1.0:1 with respect to the drug.

In another embodiment, the coating formulation includes a vasoconstrictor, which can comprise, without limitation, amidephrine, cafaminol, cyclopentamine, deoxyepinephrine, epinephrine, felypressin, indanazoline, metizoline, midodrine, naphazoline, nordefrin, octodrine, ornipressin, oxymethazoline, phenylephrine, phenylethanolamine, phenylpropanolamine, propylhexedrine, pseudoephedrine, tetrahydrozoline, tramazoline, tuaminoheptane, tyrnazoline, vasopressin, xylometazoline and the mixtures thereof. The most preferred vasoconstrictors include epinephrine, naphazoline, tetrahydrozoline indanazoline, metizoline, tramazoline, tymazoline, oxymetazoline and xylometazoline. The concentration of the vasoconstrictor, if employed, is preferably in the range of approximately 0.1 wt % to 10 wt % of the coating formulation.

In another embodiment of the invention, the coating formulation includes at least one “pathway patency modulator”, which can comprise, without limitation, osmotic agents (e.g., sodium chloride), zwitterionic compounds (e.g., amino acids), and anti-inflammatory agents, such as betamethasone 21-phosphate disodium salt, triamcinolone acetonide 21-disodium phosphate, hydrocortamate hydrochloride, hydrocortisone 21-phosphate disodium salt, methylprednisolone 21-phosphate disodium salt, methylprednisolone 21-succinate sodium salt, paramethasone disodium phosphate and prednisolone 21-succinate sodium salt, and anticoagulants, such as citric acid, citrate salts (e.g., sodium citrate), dextrin sulfate sodium, aspirin and EDTA.

In yet another embodiment of the invention, the coating formulation includes a solubilising/complexing agent, which can comprise Alpha-Cyclodextrin, Beta-Cyclodextrin, Gamma-Cyclodextrin, glucosyl-alpha-Cyclodextrin, maltosyl-alpha-Cyclodextrin, glucosyl-beta-Cyclodextrin, maltosyl-beta-Cyclodextrin, hydroxypropyl beta-Cyclodextrin, 2-hydroxypropyl-beta-Cyclodextrin, 2-hydroxypropyl-gamma-Cyclodextrin, hydroxyethyl-beta-Cyclodextrin, methyl-beta-Cyclodextrin, sulfobutylether-alpha-Cyclodextrin, sulfobutylether-beta-Cyclodextrin, and sulfobutylether-gamma-Cyclodextrin. Most preferred solubilising/complexing agents are beta-Cyclodextrin, hydroxypropyl beta-Cyclodextrin, 2-hydroxypropyl-beta-Cyclodextrin and sulfobutylether7 beta-Cyclodextrin. The concentration of the solubilising/complexing agent, if employed, is preferably in the range of approximately 1 wt. % to 20 wt. % of the coating formulation.

In another embodiment of the invention, the coating formulation includes at least one non-aqueous solvent, such as ethanol, isopropanol, methanol, propanol, butanol, propylene glycol, dimethysulfoxide, glycerin, N,N-dimethylformamide and polyethylene glycol 400. Preferably, the non-aqueous solvent is present in the coating formulation in the range of approximately 1 wt % to 50 wt % of the coating formulation. Other known formulation adjuvants can also be added to the coating formulations provided they do not adversely affect the necessary solubility and viscosity characteristics of the coating formulation and the physical integrity of the dried coating.

In one embodiment of the invention, the thickness of the biocompatible coating (drug coating) is less than 25μ, more preferably, less than 10μ, as measured from the microprojection surface. The desired coating thickness is dependent upon several factors, including the required dosage and, hence, coating thickness necessary to deliver the dosage, the density of the microprojections per unit area of the sheet, the viscosity and concentration of the coating composition and the coating method chosen.

In accordance with one embodiment of the invention, the method for delivering a drug contained in the biocompatible coating on the microprojection member includes the following steps: the coated microprojection member is initially applied to the patient's skin via an actuator, wherein the microprojections pierce the stratum corneum. The coated microprojection member is preferably left on the skin for a period lasting from 5 seconds to 24 hours. Following the desired wearing time, the microprojection member is removed.

The drug coating can be formed on microprojections by using rollers, for example, with the method and apparatus described by US patent publication 20020132054, which in incorporated by reference herein in its entirety. Briefly described, a coating liquid containing a drug is conveyed to a liquid holding surface having a coating transfer region, such as a surface of a rotating drum. A microprojection member having a microprojection array is passed over the coating transfer region such that the microprojections dip their top portions into the coating liquid at the desired depth. The depth of the coating liquid at the coating transfer region is controlled so that right amount of drug coating liquid is deposited on the microprojection at the right height on the microprojection. The depth of the coating liquid at the coating transfer region can be controlled, for example, by using a doctor blade.

After a liquid drug coating has been deposited on the microprojections, the liquid drug coating is dried to solidify the liquid drug coating. The drying can be done at ambient (room) conditions. Further, various drying techniques can be used, such as using heat, controlled lower vapor pressure of the solvent in atmosphere above the liquid, etc.

The microprojection array can be applied on the skin of an individual, for example, by using an applicator, as done with other conventional microprojection arrays.

### EXAMPLES

Below are examples of specific embodiments for carrying out the present invention. The examples are offered for illustrative purposes only, and are not intended to limit the scope of the present invention in any way.

### Example 1

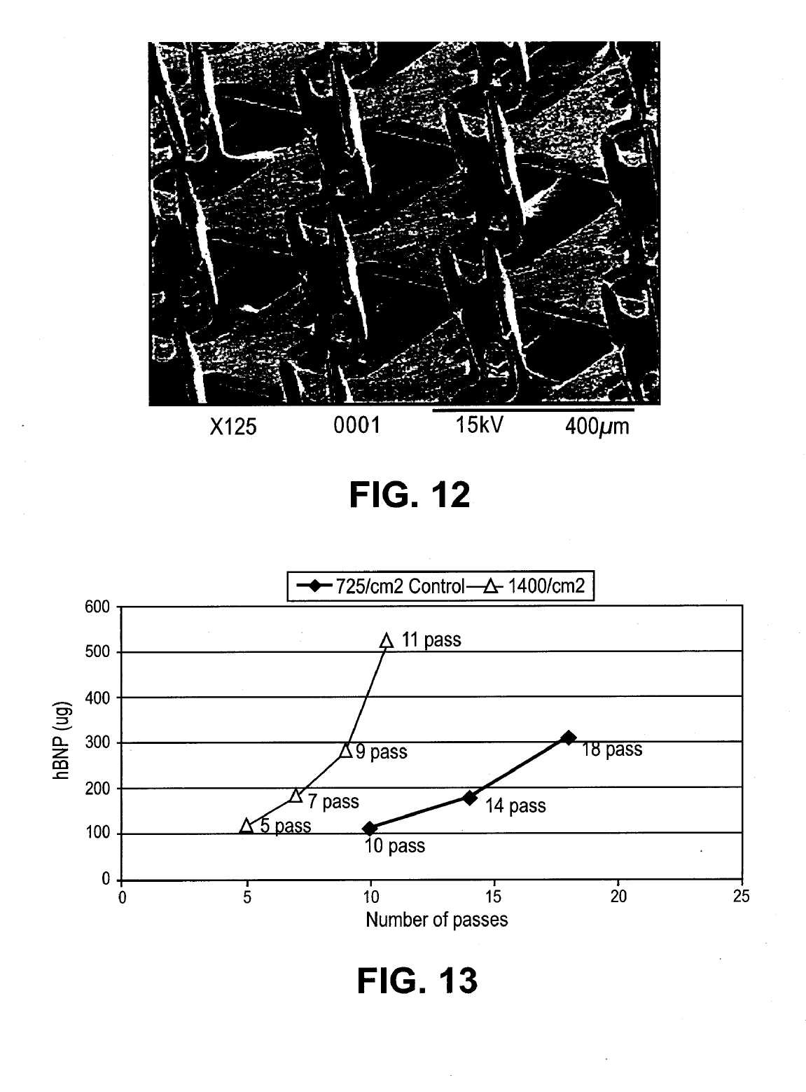

FIG. 12 shows a photograph of an microprojection array having microprojection pairs with drug coating, made by stacking two layers of microprojections together wherein the microprojections of the bottom base layer protrude through the window openings in the top microprojection base layer. The microprojection member was made by chemically etching a titanium substrate to obtain microblade arrays 2 cm² in size and 25 μm thick with methods known in the art to form arrowheaded microblades and stacking two microblade arrays to form a microprojection member.

A first substrate titanium sheet a little thicker than 25 μm was coated with photoresist, imaged for a pattern to form microblades and chemically etched with an etching solutions, such as ferric chloride solution, known in the art. The patterned polymer layer protected portions of the substrate and left other portions unprotected. After etching, the part of the substrate that was not protected by the patterned polymer layer was corroded, forming a patterned substrate having microblades that lay flat along the plane of the substrate. The microblades were then cleaned and bent using dies. This resulted in a perpendicularly extending top portion of about 225 μm length, 116 μm width, 25 μm thickness. This formed the top microprojection layer with a microblade array (the first microblade array). The top microprojection layer had a microproblade density of 725/cm². A microblade in the top microprojection layer had a planar surface area of about 5.8×10⁻³ mm². In a similar way, a bottom microprojection layer was formed to result in microproblades (microprojections) with perpendicularly extending top portion of about 250 μm length, 116 μm width, and 25 μm thickness. This formed the bottom microprojection layer with a microblade array (the second microblade array). In this way, when stacked to pair the microblades, the microblade from the bottom layer would match the microblade from the top layer at their tips. The patterns of the two layers were designed such that the windows of the two layers about coincided when the microprojections of the bottom layer protruded through the windows of the top layer matching with the top microprojections with an offset gap of about 40 μm within a pair of matched microprojections in the fashion of FIG. 11. As can be seen in FIG. 12, the planar portions associated with the microblades in a pair extended along the plane of the microprojection layers in the same direction. The edges of the two layers were aligned and affixed together by thermal fusion (welding).

The top portions of the microprojections in the microprojection member were coated with a drug formulation by dip coating with multiple passes and dried so that the liquid drug formulation solidified, using standard dip coating method known in the art, see U.S. Pat. No. 6,855,372, entitled “Method for Coating Skin Piercing Microprojections”. A drug coating known in the art can be used, e.g., those disclosed in US Patent Publications 20020132054, 20050256045. (For example, US Patent Publication 20020132054 discloses drug coatings with human growth hormone and US Patent Publication 20050256045 discloses drug coatings with parathyroid hormone.) Meniscus was seen on the bottom and on the top of the drug coating held between the microblades in the pair.

### Example 2

A first microprojection member with a single base layer was made with the method of Example 1, similar to the top microblade array of Example 1. A second microprojection member with two base layers was made in the fashion of FIG. 12, similar to the double layered microprojection member with two microblade arrays stacked in Example 1. In the second microprojection member, the microblades (microprojections) of the bottom layer protruded through the top layer and paired with corresponding microblades (microprojections) of the top layer. The top microblade array had a microblade (microprojection) density of about 725/cm². The microblades of the top layer had a perpendicularly extending top portion of 225μ length 116μ width 25μ thickness, and a planar surface area of about 5.8×10⁻³ mm². The bottom layer of microblades had a perpendicularly extending top portion of about 250μ length, 116μ width, 25μ thickness, and a planar surface area of about 5.8×10⁻³ mm². When stacked together, the tips of the microblades from the bottom layer and from the top layers are about even in distance from the layers. The two-layered microprojection member had a microprojection density of about 1400/cm². The gap between the microprojections in a pair was about 100μ. The microprojections from the first single layered microprojection member and from the second (double layered) microprojection member were each coated with a coating formulation of the drug hBNP (human brain-type Natriuretic peptide, NATRECOR made by Scios) with 25% hBNP (w/w), 6.25% sucrose (w/w), 0.10% polysorbate 20 (w/w) using standard dip coating method known in the art. The dip coating was done with multiple passes. The process was repeated so that samples with different number of dip coatings were analyzed for drug content on the microprojections. The drug coatings were analyzed by HPLC. FIG. 13 is a graph showing the drug content of the two microprojection members (one double layered and one single layered) of equal overall microprojection member planar surface after a number of passes in dip coating. The curve on the right with the diamond shaped data symbols shows the data for the singled layer microprojection members having 725 microprojections/cm². The curve on the left with triangular data symbols shows the data for the two layered microprojection members with 1400 microprojections/cm². The graph shows that the microprojection member with two layers stacked together had substantially higher drug content than the microprojection member with a single layer. In fact, the drug content of the two-layered microprojection member was more than double that of the single layered microprojection member for the same number of passes due to the presence of drug coating bridges between the microprojections in the pairs.

### Example 3

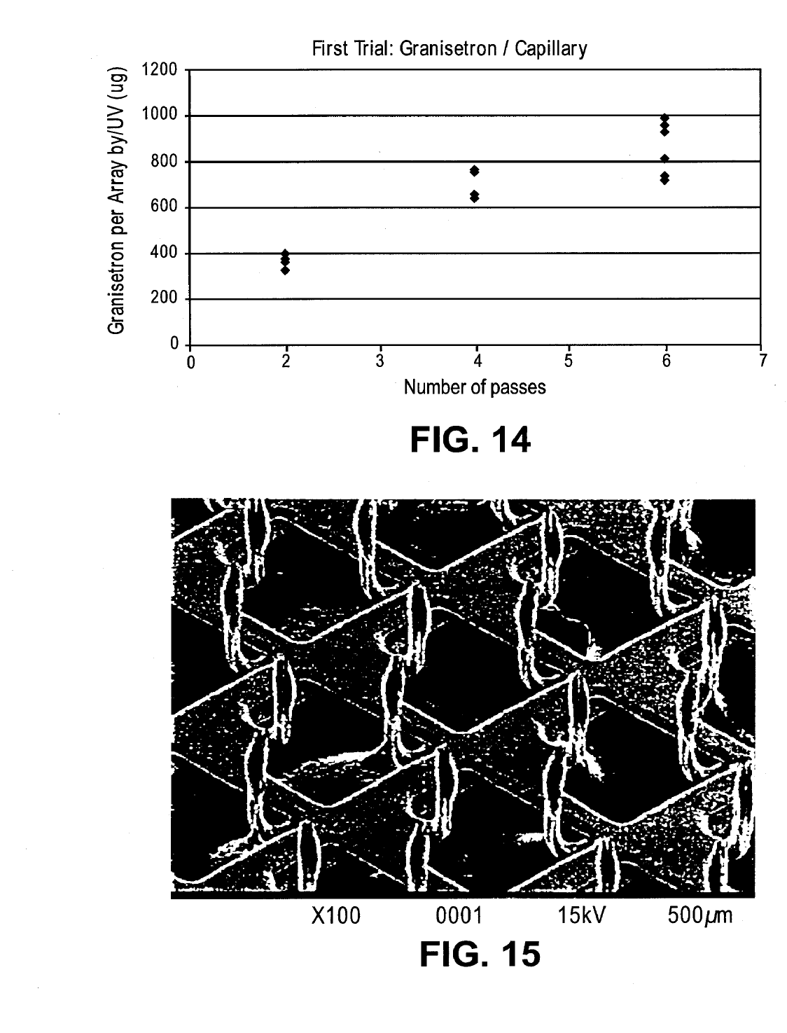

A microprojection member was made with two microprojection layers stacked together with a process similar to that described in Example 1. The microprojections of the bottom layer protruded and paired with a corresponding microprojection of the top layer. The two-layered microprojection member had a microprojection density of about 1400/cm². The gap between the microprojections in a pair was about 40μ. The two microprojection members were each coated with a coating formulation with the drug granisetron with sucrose and polysorbate similar to Example 2 using standard dip coating method known in the art. The dip coating was done with multiple passes. The process was repeated so that samples with different number of dip coatings were analyzed for drug content on the microprojections. The drug coatings were analyzed by HPLC. FIG. 14 showed the drug granisetron content of the two layered microprojection members after a number of passes in dip coating. The data points corresponding to each number of passes show the data for a few samples at the specified number of passes. The data of FIG. 14 show that the two-layered microprojection member was able to hold a significant amount of granisetron (averaged about 900 μg) after only 6 passes, significantly more than prior devices without paired microprojections in close proximity and double layered microprojection member. In a similar device with only a single-layered microprojection member, after 8 passes of dip coating, the amount of granisetron picked up by the microprojection member would have been about less than 100 μg.

### Example 4

FIG. 15 shows another embodiment of a double layered microprojection member in portion. In this example, a double layered microprojection member was made with the method similar to that of Example 1. In this microprojection member, as can be seen in FIG. 10 and FIG. 15, the microblades in the two base layers were designed such that the planar portions associated with the microblades in a pair extended along the plane of the microprojection layers in opposite direction. The microblades of each base layer were dip coated with a drug coating before the layers were stacked and affixed together.

The entire disclosure of each patent, patent application, and publication cited or described in this document is hereby incorporated herein by reference. The practice of the present invention will employ, unless otherwise indicated, conventional methods used by those in pharmaceutical product development within those of skill of the art. Embodiments of the present invention have been described with specificity. The embodiments are intended to be illustrative in all respects, rather than restrictive, of the present invention. It is to be understood that various combinations and permutations of various constituents, parts and components of the schemes disclosed herein can be implemented by one of skill in the art.

What is claimed is:

1. An apparatus for stratum-corneum piercing drug delivery, comprising: a microprojection member having a plurality of stratum corneum piercing microprojections for piercing stratum corneum to facilitate drug delivery, the microprojection member having a multilayered base member supporting microprojections, the multilayered base member having at least a first base layer and a second base layer, the first base layer supporting a first plurality of microprojections, the second base layer supporting a second plurality of microprojections, wherein the first plurality of microprojections and the second plurality of microprojections form a microprojection array for piercing the stratum corneum.

2. The apparatus of claim 1 wherein the first base layer has openings through which microprojections from the second plurality of microprojections can protrude.

3. The apparatus of claim 2 wherein the first base layer is integral and continuous with the first plurality of microprojections and the second base layer is integral and continuous with the second plurality of microprojections.

4. The apparatus of claim 2 comprising a drug coating on at least some of the microprojections from the first plurality of microprojections and a drug coating on at least some of the microprojections from the second plurality of microprojections.

5. The apparatus of claim 2 wherein at least one of the first plurality of microprojections and at least one of the second plurality of microprojections have microprojections that extend at a non-perpendicular angle from their corresponding base layer.

6. The apparatus of claim 2 wherein the microprojections of the first plurality of microprojections have lengths different from the microprojections of the second plurality of microprojections.

7. The apparatus of claim 2 wherein the first base layer and the second base layer each have openings such that openings of the first base layer match openings of the second base layer to form openings void of base layer material if viewed from a line normal to a plane of the first base layer.

8. The apparatus of claim 2 wherein the first base layer and the second base layer are stacked together in contact.

9. The apparatus of claim 8 wherein the first base layer and the second base layer are rigidly secured together.

10. The apparatus of claim 9 wherein the first base layer and the second base layer are interference fit together to secure the first base.

11. The apparatus of claim 2 wherein a first drug coating containing a first drug coats at least some of the microprojections from the first base layer and a second drug coating containing a second drug coats at least some of the microprojections from the second base layer.

12. An apparatus for stratum-corneum piercing drug delivery, comprising: a microprojection member having a plurality of stratum corneum piercing microprojections for piercing stratum corneum to facilitate drug delivery, the microprojection member having a multilayered base member supporting microprojections, the multilayered base member having at least a first base layer and a second base layer, the first base layer of the same continuous material with and integrally supporting a first plurality of microprojections, the second base layer of the same continuous material with and integrally supporting a second plurality of microprojections, the first plurality of microprojections neighboring to the second plurality of microprojections forming groups of the two pluralities of microprojections in a microprojection array for piercing the stratum corneum, a drug coating coats at least portion of the microprojection member.

13. The apparatus of claim 12 wherein the first base layer has openings through which microprojections from the second plurality of microprojections can protrude and whereby microprojections from the first plurality of microprojections pair with adjacent microprojections from the second plurality of microprojections, thereby forming a pattern of pairs in the microprojection array.

14. The apparatus of claim 13 wherein, the first plurality of microprojections extend from the first base layer at a first angle, and the second plurality of microprojections extend from the second base layer at a second angle.

15. The apparatus of claim 14 wherein the first plurality of microprojections extend from the first base layer at a 90° angle, and the second plurality of microprojections extend from the second base layer at a 90° angle.

16. The apparatus of claim 14 wherein at least one of the first angle and the second angle is non-perpendicular such that in a pair the microprojections are closer at their tips than at the base layers.

17. The apparatus of claim 14 wherein in a pair the microprojections converge at their tips forming a pinnacle.

18. The apparatus of claim 12 wherein at least some of the microprojections from the first plurality of microprojections and at least some of the microprojections from the second plurality of microprojections form groups and in a group at least one of the microprojections leans towards another microprojection.

19. The apparatus of claim 18 wherein a drug coating bridges the microprojections in the group with a meniscus.

20. The apparatus of claim 13 wherein in a pair of microprojections a continuous drug coating coats both microprojections the pair.

21. The apparatus of claim 13 wherein in a pair a continuous drug coating coats the microprojections from the first plurality of microprojections and the microprojections from the second plurality of microprojections forming a bridge of drug coating near the tips of the microprojection pair.

22. A method for stratum-corneum piercing drug delivery to an individual comprising: (1) providing a microprojection member having a multilayered base member supporting microprojections, the base member having at least a first base layer and a second base layer, the first base layer supporting a first plurality of microprojections, the second base layer supporting a second plurality of microprojections, the first plurality of microprojections and the second plurality of microprojections forming a microprojection array for piercing the stratum corneum, and (2) piercing the stratum corneum of said individual with the microprojection array.

23. The method of claim 22 comprising providing openings in the first base layer wherein microprojections from the second plurality of microprojections can protrude through said openings.

24. The method of claim 23 comprising providing a base layer that is integral and continuous with a plurality of microprojections.

25. The method of claim 22 comprising providing a microprojection array wherein at least one of the microprojections has a shaft.

26. The method of claim 25 comprising providing a microprojection having a shaft from one base layer wherein said microprojection is pointing toward a microprojection of another base layer.

27. The method of claim 22 comprising providing openings in the first base layer and the second base layer such that the openings in the first base layer match the openings in the second base layer to form openings void of base layer material if viewed from a line normal to a plane of the first base layer.

28. The method of claim 22 comprising stacking together the first base layer and the second base layer.

29. The method of claim 28 comprising rigidly securing the first base layer and the second base layer together.

30. The method of claim 22 comprising extending at least some of the microprojections at a non-perpendicular angle from their corresponding base layer.

31. The method of claim 22 comprising coating at least some of the microprojections from the first base layer and at least some of the microprojections from the second base layer with a drug coating.

32. A method for forming a stratum-corneum piercing drug delivery apparatus, comprising: forming a microprojection member having a microprojection array, the microprojection member having a multilayered base member supporting microprojections, the base member having at least a first base layer and a second base layer, the first base layer supporting a first plurality of microprojections, the second base layer supporting a second plurality of microprojections, the first plurality of microprojections and the second plurality of microprojections forming a microprojection array for piercing the stratum corneum.

33. The method of claim 32 comprising forming openings on the first base layer and extending at least some of the microprojections from the second plurality of microprojections to protrude through said openings.

34. The method of claim 33 comprising forming a microprojection member wherein the first base layer is integral and continuous with the first plurality of microprojections and the second base layer is integral and continuous with the second plurality of microprojections.

35. The method of claim 32 comprising forming microprojections in at least one of the first plurality of microprojections and the second plurality of microprojections such that a resultant microprojection has a shaft portion that points to a microprojection of another base layer.

36. The method of claim 32 comprising stacking the first base layer and the second base layer together in contact.

37. The method of claim 32 comprising aligning and stacking the first base layer and the second base layer

38. The method of claim 37 comprising rigidly securing the first base layer and the second base layer together.

39. The method of claim 32 comprising coating at least some of the microprojections from the first base layer and at least some of the microprojections from the second base layer.
